(19) United States
(12) Patent Application Publication
(10) Pub. No.: US 2012/0310224 A1
(43) Pub. Date: Dec. 6, 2012

(54) OPHTHALMIC LASER SURGERY APPARATUS

(75) Inventor: Tomohiro MIYAGI, Toyokawa (JP)

(73) Assignee: NIDEK CO., LTD., Gamagori (JP)

(21) Appl. No.: 13/483,562

(22) Filed: May 30, 2012

(30) Foreign Application Priority Data
May 31, 2011 (JP) 2011-122841

(51) Int. Cl. A61F 9/01

(52) U.S. Cl. 606/5

(57) ABSTRACT

An ophthalmic laser surgery apparatus for cutting a tissue in a cornea with laser light into a lenticular tissue corresponding to an amount of correction of refractive-error, includes: a pulse laser light source for outputting pulse laser light; an irradiation optical system including a moving optical system for moving a laser spot to which laser light is collected; a setting unit configured to set a cutting line for cutting the lenticular tissue to have a cut tissue by laser light irradiation such that the further cut lenticular tissue has a width equal to or less than a half of a diameter of the lenticular tissue, and that the further cut lenticular tissue is interlinked together without being separated from one another; and a control unit configured to drive the moving optical system based on the set cutting line and to cut a corneal tissue with laser light.

### CROSS REFERENCE TO RELATED APPLICATION

This application is based upon and claims the benefit of priority of Japanese Patent Application No. 2011-122841 filed on May 31, 2011, the contents of which are incorporated herein by reference in its entirety.

### BACKGROUND

The present invention relates to an ophthalmic laser surgery apparatus for operating a patient's eye (an operative eye) by irradiating laser light thereonto.

An apparatus is known, which cuts tissues such as a cornea of a patient's eye (an operative eye) by irradiating ultrashort pulse laser beams such as femtosecond pulse laser beams thereonto (see, e.g., JP-T-2011-507559). An apparatus according to JP-T-2011-507559 cuts, with laser light whose spot is minute, a corneal tissue into a lenticular shape corresponding to an amount of correction of refractive-error. The shape of a corneal surface is changed by taking out a lenticular tissue from an incisional wound formed on a cornea. Thus, the correction of refractive-error is performed.

### SUMMARY

However, because a lenticular tissue is extracted in an operation without being changed, it is necessary to form a relatively large incisional wound on the cornea. If the incisional wound is large, the possibility of induction of postoperative corneal-astigmatism is increased. In order to suppress the postoperative change of the corneal shape, it is desired that the incisional wound is small, and that burden to the incisional wound is small.

A problem that the invention solves is to provide an ophthalmic surgery apparatus capable of making, as small as possible, an incisional wound formed on an eyeball to extract an eyeball tissue therefrom.

(1) An ophthalmic laser surgery apparatus for cutting a tissue in a cornea with laser light into a lenticular tissue corresponding to an amount of correction of refractive-error, the ophthalmic laser surgery apparatus comprising:

a pulse laser light source configured to output pulse laser light for causing breakdown at a laser spot;

an irradiation optical system including a moving optical system for three-dimensionally moving a laser spot to which laser light is collected;

a setting unit configured to set a cutting line for further cutting the lenticular tissue into a cut tissue by laser light irradiation such that the cut tissue has a width equal to or less than a half of a diameter of the lenticular tissue, and that the cut tissue is interlinked together without being separated from one another; and

a control unit configured to drive the moving optical system based on the set cutting line and to cut a corneal tissue with laser light.

(2) The ophthalmic laser surgery apparatus according to (1), wherein

the setting unit includes a storage unit configured to store a cutting pattern having a cutting line according to a predetermined rule, and

the setting unit sets, based on the cutting pattern, a cutting line such that a width of the cut tissue is equal to or less than a half of the diameter of the lenticular tissue.

(3) The ophthalmic laser surgery apparatus according to (2), wherein the cutting pattern is a pattern having a cutting line configured such that the cut tissue is a spiral tissue.
(4) The ophthalmic laser surgery apparatus according to (2), wherein the cutting pattern is a pattern having a cutting line configured such that both ends of the cut tissue are located on an outer periphery of the lenticular tissue and that the cut tissue has a zigzag-shape.
(5) The ophthalmic laser surgery apparatus according to (2), wherein

the setting unit includes an input unit configured to input a width of an incisional wound formed on the cornea to take out the cut tissue to outside of the cornea, and a diameter of the lenticular tissue,

a width of the cut tissue which is cut based on the input width of the incisional wound is determined to have a value equal to or less than a half of the diameter of the lenticular tissue, and

the cutting line is set based on the determined width and the cutting pattern.

### BRIEF DESCRIPTION OF THE DRAWINGS

FIG. 1 is a schematic configuration view illustrating an ophthalmic laser surgery apparatus that is an embodiment of the invention.

FIGS. 2A and 2B are explanatory views illustrating a corneal tissue cut into a lenticular shape by conventional laser irradiation.

FIGS. 3A and 3B are explanatory views illustrating patterns having lines for further cutting the lenticular tissue, based on laser irradiation patterns according to the invention.

FIG. 4 is an explanatory view illustrating the screen of a monitor.

FIG. 5 is an explanatory view illustrating an example of modification of the pattern of a cutting line.

FIG. 6 is an explanatory view illustrating a pattern for cutting a corneal tissue into a zigzag shape.

FIG. 7 is an explanatory view illustrating another example of modification of the pattern illustrated in FIG. 6.

### DESCRIPTION OF EXEMPLARY EMBODIMENTS

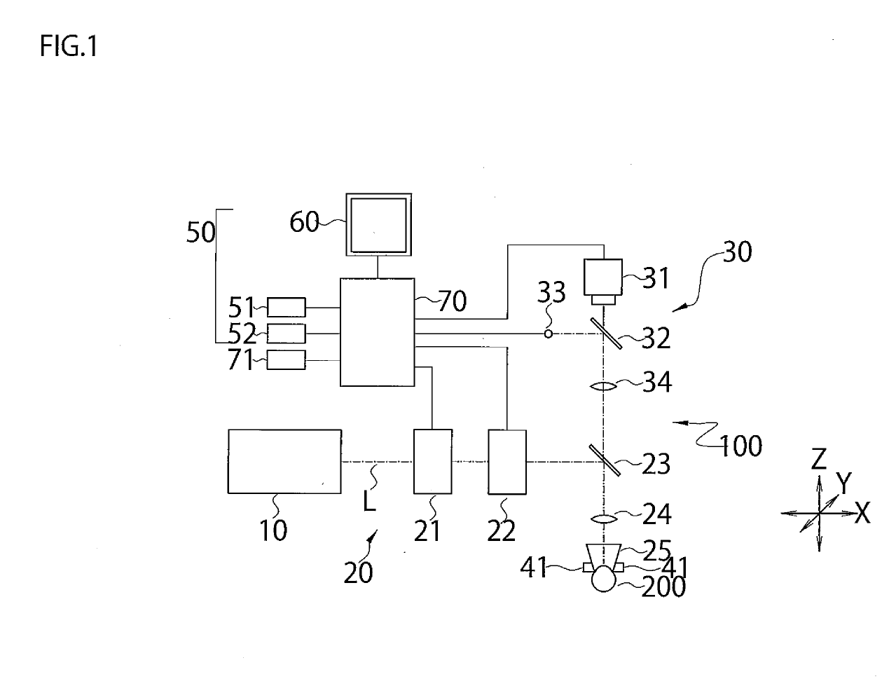

Hereinafter, an exemplary embodiment of the invention will be described with reference to the drawings. FIG. 1 is a schematic configuration view illustrating an ophthalmic laser surgery apparatus according to the exemplary embodiment. Incidentally, the apparatus according to the exemplary embodiment is for processing and cutting eyeball tissues (a cornea, a crystalline lens, etc.) of an operative eye (a patient's eye) which is a processing-object to be processed.

An ophthalmic laser surgery apparatus 100 includes a laser unit (a laser light source) 10 for outputting laser light pulses having a characteristic of causing a breakdown at a laser spot, a laser irradiation optical system (a laser irradiation unit) 20 for guiding laser light and irradiating the laser light onto a target (an eyeball tissue), an observation optical system (an observation unit) 30 for observing an operative eye, an eyeball fixing unit 40 for fixing and holding the operative eye, an operation unit 50 for operating the apparatus 100, a monitor 60 serving as a display means for setting, checking and etc. of the apparatus 100, and a control portion 70 for supervising and controlling the apparatus 100.

The laser unit 10 is a laser light source for outputting ultrashort pulse laser light beams that generate plasma (i.e., causing breakdown) at a laser-light-collecting point (i.e., a laser spot). A device for outputting pulse laser beams whose pulse width is of femtosecond order to pico-second order is used as the laser unit 10. Plasma is generated at the pulse-laser-light-collecting point. A target tissue, i.e., a corneal tissue in this case is cut off.

The irradiation optical system 20 includes a scanning portion (an optical scanner) 21 for scanning (or deflecting) a pulse-laser-light beam spot on a target surface two-dimensionally (i.e., in X- and Y-directions), a focal point moving portion (i.e., a Z-scanner) 22 for moving a pulse-laser-light beam spot in a depth direction (i.e., in Z-direction), a beam splitter 23 for aligning the optical axis of laser light with an observation optical axis of the observation optical system 30, an object lens 24 serving as an image-formation optical system for causing laser light to form an image on the target surface, and an applicator 25 to be contacted with the operative eye. The scanning portion 21 and the focal point moving portion 22 configure a movement optical system adapted to cause a laser-light beam spot to move three-dimensionally in a corneal tissue.

Two galvanomirrors whose rotation axes are orthogonal to each other are used as the scanning portion 21. Consequently, a laser-light beam spot is scanned on a predetermined two-dimensional plane (i.e., an XY-plane). Incidentally, a scanner, etc. configured of a combination of a resonantmirror, a rotating prism, a polygonmirror, and a galvanomirror can be used as the scanning portion. The focal point moving portion 22 includes an optical element arranged upstream (i.e., at the side of the laser unit 10 of) the object lens 24. The position of the laser-light beam spot is moved along the optical axis (i.e., along Z-direction) by moving the optical element of the focal point moving portion 22 along the optical axis. The beam splitter 23 is set to be a dichroic mirror having properties set to reflect laser light and to transmit illuminating light and reflection light of the illuminating light. The object lens 24 serves to cause pulse-laser-light to form an image on the target surface such that the pulse-laser-light has a minute spot diameter of micron-order to submicron-order. The applicator 25 is a (transparent) contact lens having translucency and is used for applanation of the cornea of an operative eye. At the applanation, the cornea of the operative eye is positioned at a certain distance from the front surface (i.e., the contact surface) of the applicator. Accordingly, the applicator 25 serves to fix and hold an eyeball tissue, and to perform the positioning of laser irradiation.

A breakdown caused at the spot position of laser light produces a mechanical fracture (such as a crack) whose size is comparable to a spot size of the laser light on the eyeball tissue. The spot of laser light is moved in X- and Y-directions by the scanning portion 21 and in Z-direction by the focal point moving portion 22. Thus, the spot is moved (i.e., changed in position) three-dimensionally. The spot of laser light is moved three-dimensionally on the eyeball tissue. The eyeball tissue is cut into a three-dimensional shape (i.e., a preset laser irradiation pattern) by connecting the spots. The laser irradiation pattern is described below in detail.

The observation optical system 30 includes a camera 31 having a two-dimensional imaging element, a beam splitter 32, an illuminating light source 33, an optical element 34 serving as a light guiding optical system for guiding illuminating light and reflection light at an operative eye. The beam splitter 32 has properties set to reflect illuminating light and transmit reflection light from the operative eye. In this case, a half mirror is used as the beam splitter 32. The illuminating light source 33 emits illuminating light, such as visible light, suitable for illuminating an operative eye. The two-dimensional imaging element of the camera 31 is, e.g., an imager having sensitivity to the wavelength of the illuminating light. The illuminating light source 33 can be a light source that outputs infrared light. Incidentally, the observation optical system 30 shares the object lens 24 with the irradiation optical system 20.

The eyeball fixing unit 40 includes a suction ring 41, and the applicator 25. The suction ring 41 is an annular member and has a shape abutting against the sclera of an anterior eye. The suction ring 41 is subjected to suction by a pump, etc. (not shown), and fixes and holds an operative eye by attracting an eyeball to the suction ring 41. Incidentally, the applicator 25 can be of the type that fills the inner space of the suction ring 41 with liquid. In this case, the space between the lens (contact lens) and a cornea is filled with the liquid. Thus, the cornea is not applanated by the lens.

The operation unit 50 includes an input means 51 for setting the apparatus 100, and a footswitch 52 for inputting a laser irradiation trigger signal. For example, a mouse serving as a pointing device for specifying operation conditions, etc. on a setting screen displayed on a monitor 60, a keyboard for inputting numerical values and information concerning operation conditions, etc., and so on are used as the input means 51.

A frontal view of an operative eye, which is taken by the camera 31, is shown on the monitor 60. In addition, the operation conditions, the laser irradiation pattern and so on are displayed on the monitor 60.

A control portion 70 is a central processing unit (CPU), to which the laser unit 10, the scanning portion 21, the focal point moving portion 22, the camera 31, and the illuminating light source 33 are connected. A memory 71 storing control programs, laser-irradiation cutting-line patterns, set operation conditions, taken images, and so on is connected to the control portion 70. The memory 71 also stores measurement data obtained by other measurement devices.

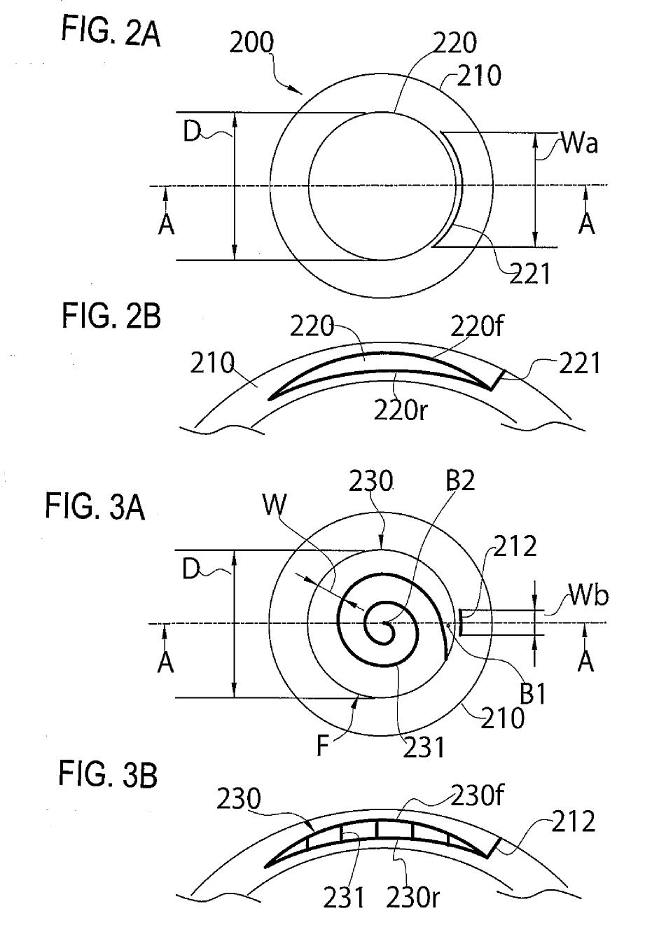

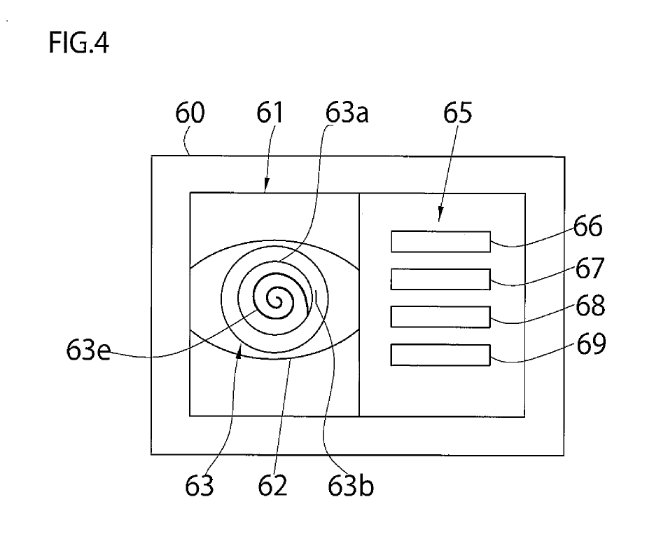

Next, the laser-irradiation cutting-line pattern (hereinafter referred to simply as the pattern) is described hereinbelow. FIGS. 2A and 2B are explanatory views illustrating a corneal tissue cut into a lenticular shape by conventional laser irradiation. FIGS. 3A and 3B are explanatory views illustrating patterns having lines for further cutting the lenticular tissue, based on laser irradiation patterns according to the invention. FIGS. 2A and 3A are front views of a cornea. Each of FIGS. 2B and 3B is a cross-sectional view taken on line A-A illustrated in an associated one of FIGS. 2A and 3A. FIGS. 2A and 3A show an XY-plane. FIGS. 2B and 3B show an XZ-plane. In this case, for convenience of description, the illustration of the applicator 25 is omitted.

FIG. 2B illustrates, with dotted lines, a lenticular tissue (i.e., a convex-lenticular tissue) 220 (hereinafter referred to as a lens 220) having capacity surrounded by a front curved surface 220f and a rear curved surface 220r in a cornea 210 of an operative eye 200. As illustrated in FIG. 2A, an edge portion (i.e., an outer peripheral circle) 220c of the lens 220 is designated with D. The curvature of (the front surface of) the cornea is changed by removing the lens 220 from the cornea 210. Consequently, the refractive power of the entire cornea 210 is changed. Accordingly, the vision of the operative eye 200 is corrected. In this case, the curvature of the front surface of the cornea 210 is increased. The refractive power of the entire cornea 210 is lowered. Thus, the myopia of the operative eye 200 is corrected. Incidentally, as illustrated in FIG. 2A, the lens 220 is taken (or pulled) out from an incisional wound 211, which is formed on the cornea 210 and has a width (an incision width) Wa, to the outside of an eyeball.

Accordingly, boundary portions (i.e., the front curved surface 220f and the rear curved surface 220r) of the lens 220 configure a pattern. The shape of the incisional wound 211 is included by the pattern. The lens 220 and the incisional wound 211 are formed by cutting the tissue with laser irradiation. The tissue is cut so as to be connected together by irradiating laser spots onto positions illustrated with the dotted lines. Thus, the lens 220 is formed in the cornea 210 in a state in which the lens 220 is separated from the cornea. Incidentally, the thickness of the lens 220 (thus, the shapes of the front curved surface 220f and the rear curved surface 220r) is determined by the power of the lens 220, which is described below.

As illustrated in FIGS. 3A and 3B, patterns according to the invention are adapted to have cutting-lines for further cutting the lens 220 illustrated in FIGS. 2A and 2B. As illustrated in FIG. 3A, the shape of a lens 230 formed in the cornea 210 is the same as that of the above lens 220. That is, the diameter of the lens 230 is D and has capacity surrounded by a front curved surface 230f and a rear curved surface 230r. The thickness of the lens 230 is equal to that of the lens 220.

As illustrated in FIG. 3A, a pattern is set, which has cutting lines 231 for further cutting the lens 230. The lens 230 is cut off according to the set cutting line 231. Each tissue of the lens 230 cut according to the cutting line 231 is made to have a width equal to or less than a half the diameter D of the lens 230. In addition, the tissue of the cut lens 230 is brought into a state in which the tissue thereof is not separated from one another and interlinked together. If the width of each tissue of the cut lens 230 is equal to or less than a half the diameter D of the lens 230, the lens 230 can be taken out from an incisional wound smaller than the conventional incisional wound Wa.

The pattern according to the present embodiment is formed of the front curved surface 230f and the rear curved surface 230r to form the lens 230, the cutting line 231 having a specific rule in the case the lens 230 is viewed from the XY-plane, and an incisional wound 212 having a width Wb. The incisional wound 212 is less than the incisional wound 211 shown in FIGS. 2A and 2B in width (incision width), so that the widths of the incisional wounds have the following relationship: Wa>Wb.

The lens 230 are cut along a cutting line 231 indicated by a dotted line. In an example of a pattern illustrated in FIG. 3A, the cutting line 231 is a single curved line interlinked on the XY-plane (i.e., a predetermined two-dimensional plane). In this case, the cutting line 231 is a spiral curved line formed by interlinking the cut tissue like a spiral-curve in the lens 230. Preferably, the tissue (i.e., the cut tissues) cut according to the cutting line 231 is configured to have a diameter (or a cross-sectional shape thereof at a pass point) sufficient to the extent that when the cut tissue is taken out of the eyeball from the incisional wound 212, the tissue passing through the incisional wound 212 is difficult to put a strain on the incisional wound 212. If the diameter of each of the cut tissue is too large in comparison with the incisional wound 212, an opening force is applied to the incisional wound 212 to thereby put a strain on the peripheral tissue of the incisional wound 212. Preferably, the cut tissue interlinked together has a diameter at which the cut tissue has strength sufficient to the extent that the interlinked state of the tissue can be maintained. If the diameter of each of the cut tissue is too small in comparison with the incisional wound 212, e.g., when the cut tissue is pulled, the cut tissue interlinked together may break halfway. This is undesirable. The pattern represented by a graphic F is stored in the memory 71.

More specifically, in a case where the lens 230 is viewed with respect to XY-plane (from the front), the lens 230 is cut off according to the cutting line 231 as a spiral graphic F. The graphic F is formed as a single element (having a unified shape) connected from a starting point (one end) on the graphic (i.e., the cut tissue) to an end point (i.e., the other end). Assuming that a point at the side of the incisional wound 212 is the starting point B, and that a point at a central portion (or midportion) of the cornea 210 is an end point, the cutting line 231 is set such that the starting point B is located in the vicinity of the incisional wound 212. Consequently, when the cut tissue is taken out, an end portion of the cut tissue can easily be held by tweezers, etc. The graphic F has a shape formed such that when the cut tissue interlinked together is taken out from the incisional wound 212, the cut tissue is suppressed from breaking halfway due to concentration of stress at a part at which the curvature of the tissue is large. Roughly, the graphic F has a width W and is shaped to swirl from the center of the lens 230 to the outer periphery thereof. Because the graphic F is spirally shaped, when the cut tissue is pulled out from the starting point B to the outside of the eyeball, the tissue connected from the starting point B is moved to the incisional wound 212 by simultaneously rotating in the cornea 210. Thus, the cut tissue can smoothly be performed. In the graphic F, the width W of the cut tissue is set to be about twice the width Wa of the incisional wound 212 or less. The width W is measured in XY-plane. Each of the actual cut tissue has a thickness in Z-direction. However, according to the present embodiment, it is advisable to set the width W to be equal to or less than twice the width Wa. This is determined according to the following conditions. Although the thickness of the lens 230 depends upon operative eyes, the thicknesses of the lenses 230 of the operative eyes are not much different from one another. Thus, the cross-sectional shape taken in Z-direction of the cut tissue interlinked together is assumed not to largely vary with operative eyes and operation conditions. Accordingly, the cutting of the lens 230 can be determined according to the width W.

Thus, the cutting line 231 is determined according to the shape of the lens 230 and the spiral pattern stored in the memory 71. Information concerning the cutting line 231 is stored in the memory 71 as information for controlling the focal point moving portion 22, and that for driving the laser unit 10. The width W of the cutting line 231 can be changed by the operation unit 50.

Next, the setting of the pattern having the cutting line 231 is described hereinafter. A frontal view display portion 61 and an operation condition setting portion 65 are displayed on the screen of the monitor 60.

A frontal view 62 of an operative eye is displayed on the frontal view display portion 61. A pattern 63 is displayed to be superimposed on the frontal view 62. The pattern 63 includes a circle 63a indicating an outer periphery of the lenticular tissue to be cut, a line 63b indicating an incisional wound, and a curved line 63c indicating a cutting line for further cutting the lenticular tissue. The circle 63a is displayed such that the center of the circle 63a is located at the light-guiding center position of the frontal view 62. The line 63b is such that a position at which the line 63b is arranged can be changed by the input means 51.

The operation condition setting portion 65 includes a diameter setting portion 66 for setting the diameter D of a lenticular tissue, a correction degree setting portion 67 for setting the degree of correction (diopter) to determine the shape of a lenticular shape, an incision width setting portion 68 for setting the width Wb of an incisional wound, and a pattern setting portion 69. Each of the setting portions is designated by the input means 51, so that numerical values, etc. are set (input). The pattern setting portion 69 is configured to be able to select plural cutting-line patterns (e.g., patterns illustrated in FIGS. 5 to 7), in addition to the spiral pattern illustrated in FIG. 3A.

When the diameter D is set by the diameter setting portion 66 and the width Wb is set by the incision width setting portion 68, the control portion 70 calculates the width W of the tissue to be cut according to the cutting line 231. The control portion 70 calculates the width W to be set as being less than a half the diameter D, preferably, as being equal to or less than twice the width Wb, and as being able to nearly uniformly divide the diameter D. The control portion 70 generates a curved line 63c indicating the cutting line 231 at which the width of each of the adjacent curved lines 63c is nearly equal to a width W. The control portion 70 displays the curved line 63c to be superimposed on the frontal view 62 so that an end of the curved line 63c is located in the vicinity of the incisional wound 63b.

Thus, the operation conditions for operating an operative eye, and the laser irradiation pattern including the cutting line are set. The set numerical values and the set cutting line of the pattern are stored in the memory 71. Incidentally, a laser output, a scan rate (i.e., the number of shots per unit time), etc. are set by a laser irradiation condition setting portion (not shown).

An operation of the apparatus having the above configuration is described hereinafter. Before an operation, an operator sets the operation conditions and the pattern. The operator causes a patient to lie on a surgical bed. Then, the operator fixes the patient's eyeball with the eyeball fixing unit 40. The operative eye is held by the suction ring 41 and is applanated by the applicator 25. When a footswitch 42 is stepped by the operator, the control portion 70 starts laser irradiation based on a trigger signal. The control portion 70 causes the laser unit to irradiate laser light onto the eye, based on the set operation conditions and the set pattern. The control portion 70 controls the laser unit 10, based on the position of the cutting line of the pattern. In addition, the control portion 70 controls the scanning portion 21 and the focal point moving portion 22. At that time, the control portion 70 causes a laser spot to move from the rear surface of the pattern to the front surface thereof. In the cornea 210, cut tissue is formed by further cutting the lens 230 according to the cutting line 231. In addition, the incisional wound 212 is formed on the cornea 210.

Upon completion of the laser irradiation, the operator inserts the tweezers into the cornea 210 from the incisional wound 212. Then, the operator holds an end (i.e., the starting point B of the graphic F) of the cut tissue with the tweezers and takes out (or extracts) the cut tissue from the incisional wound 212.

Thus, the lens 230 is removed from the cornea 210. The refractive power of the cornea 210 is changed, so that the vision of the operative eye 200 is corrected. At such an operation, the lens 230 is cut off. When the lens 230 is passed through the incisional wound 212, the cross-sectional shape of the lens 230 is small. Thus, the lens 230 is difficult to put a strain on the incisional wound 212. In addition, the width Wb of the incisional wound 212 can be reduced by decreasing the size of the tissue to be extracted. Consequently, the possibility of induction of postoperative corneal-astigmatism is reduced. The lens 230 is a unified tissue. Thus, the entire tissue can be removed by pulling out an end portion of the cut tissue from the incisional wound 212. Accordingly, an operation time can be reduced.

Although FIGS. 3A and 3B illustrate a convex lens for myopic correction as an example of the lens 230, a concave lens therefor can be used as the lens 230. In addition, in the case of astigmatism correction, the shape of the cut tissue interlinked together is set to have a cylindrical component.

In the above description, the spiral cutting-line pattern for spirally cutting the corneal tissue of an eyeball has been exemplified. The cutting-line pattern according to the invention is not limited thereto. Any other cutting-line patterns can be used, as long as the width of the cut tissue interlinked together is equal to or less than a half the diameter of the lens 230, and the cut tissue is brought into a state in which the tissue is not separated from one another and interlinked together. FIGS. 5 to 7 are views illustrating examples of modification of the cutting-line pattern.

In an example illustrated in FIG. 5, the cutting line 231 is set according to a rule that defines the cutting line as a single straight line extending from the outer periphery of the lens 230 to the center. In the case of this example, the width W of the cut tissue is set according to the cutting line 231 to be a half the diameter D of the lens 230. The length of the cutting line 231 is determined, based on the input diameter D of the lens 230. In addition, the direction of the cutting line 231 is determined by preliminarily inputting the position of the incisional wound 212. Even in the case of this example, at least the width of the incisional wound 212 can be reduced, as compared with the conventional example illustrated in FIG. 2A.

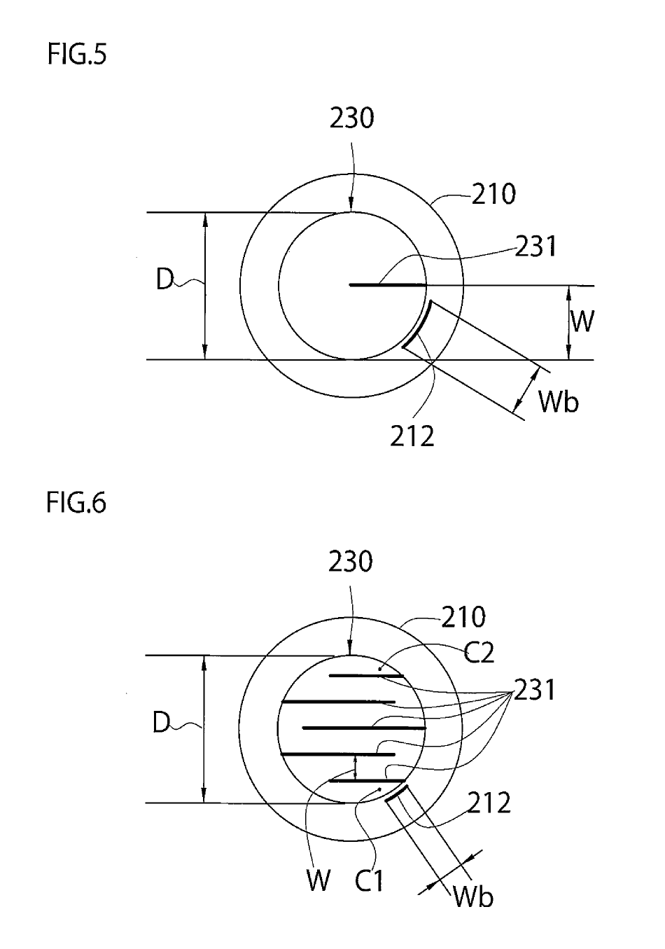

FIG. 6 illustrates an example of further reducing the width W of the cut tissue, as compared with the example illustrated in FIG. 5. That is, the example illustrated in FIG. 6 is an example of a pattern according to a rule that this pattern has plural cutting lines 231 for cutting the corneal tissue into a zigzag shape. In the case of this example, both ends C1 and C2 of the interlinked corneal tissue are located on the outer periphery of the lenticular tissue 230. The width Wb of the incisional wound 212 is input to thereby determine the width W as being equal to or less than twice the width Wb. Preferably, the width W is determined to be substantially equal to the width Wb. The number of the cutting lines 231, and the length of each cutting line 231 can be determined by determining the width W and inputting the diameter D of the lens 230. If the position of the incisional wound 212 is first set, the direction of each cutting line 231 is determined, based on the position of the incisional wound 212. That is, the direction of each cutting line 231 is determined so that the end C1 or C2 of the corneal tissue interlinked together is located in the vicinity of the incisional wound 212. Incidentally, the width W can optionally be specified by the input portion.

In the example illustrated in FIG. 6, the width W is set to be small, as compared with the example illustrated in FIG. 5. Thus, the width Wb of the incisional wound 212 can be further reduced.

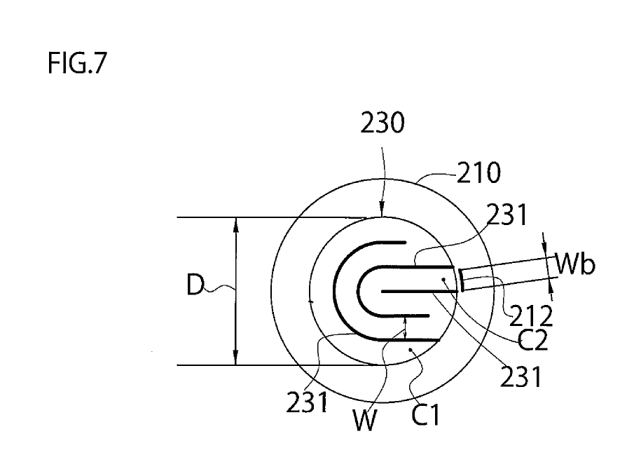

FIG. 7 illustrates another modification of the zigzag pattern illustrated in FIG. 6. FIG. 6 illustrates a zigzag pattern using straight cutting lines. On the other hand, FIG. 7 illustrates a pattern having curved cutting lines extending along the outer periphery of the lens 230 as a part of the cutting lines. Even in the pattern illustrated in FIG. 7, i.e., even in this example, the width W is determined by inputting the width Wb of the incisional wound 212 (alternatively, the width W is optionally input). Then, the number of cutting lines 231, and the length of each cutting line 231 are determined, based on the width W and the diameter D of the lens 230. The direction of each cutting line 231, and the positions of both ends C1 and C2 of the corneal tissue interlinked together are determined based on the position of the incisional wound 212. Alternatively, the direction of each cutting line 231, and the positions of both ends C1 and C2 are optionally set.

Incidentally, in the above description, the apparatus is configured to set the pattern to be a single tissue (or graphic) from which the lens 230 is not separated. Any other pattern can be employed, as long as a strain put on the incisional wound can be reduced when the eyeball tissue is extracted, or as long as the size of the incisional wound can be reduced. The apparatus can be configured to set a pattern for dividing the tissue (the lens 230) into two or more small pieces. In this case, the divided tissue is formed of subpatterns that maintain a linked state without being separated from one another. For example, a lens is divided into two subpatterns. A pattern including the two subpatterns is set to be the cutting-line pattern. Spiral patterns can be cited as the subpatterns. The cutting-line pattern is set so that both the first and second subpatterns are spiral and engage with each other. In this case, e.g., the apparatus can be configured in the following manner. That is, two incisional wounds are formed at opposed positions on the cornea. The tissue cut according to the first subpattern, and that cut according to the second subpattern are taken out from the incisional wounds, respectively. (Thus, the cutting-line pattern can be configured in such a manner.) Accordingly, the incision width of each single incisional wound can be reduced. Alternatively, the apparatus can be configured such that the tissue cut according to a cutting-line pattern including plural subpatterns is taken out from a single incisional wound.

Although the above embodiment uses a femtosecond pulse type optical laser as a laser, the laser unit used by the apparatus according to the invention is not limited thereto. Any other laser can be employed, as long as the laser has properties such that fine processing of micron-order on a processing-object can be performed without heating, regardless of the material of the processing-object, that internal-processing of a transparent processing-object can be performed, and that ultrashort pulse laser beams, such as pico-second pulse laser beams, can be emitted therefrom.

As described above, the invention is not limited to the embodiments. Various modifications can be made to the invention, and the modifications are also included in the invention within the range of the same technical idea.

What is claimed is:

1. An ophthalmic laser surgery apparatus for cutting a tissue in a cornea with laser light into a lenticular tissue corresponding to an amount of correction of refractive-error, the ophthalmic laser surgery apparatus comprising:
a pulse laser light source configured to output pulse laser light for causing breakdown at a laser spot;
an irradiation optical system including a moving optical system for three-dimensionally moving a laser spot to which laser light is collected;
a setting unit configured to set a cutting line for further cutting the lenticular tissue into a cut tissue by laser light irradiation such that the cut tissue has a width equal to or less than a half of a diameter of the lenticular tissue, and that the cut tissue is interlinked together without being separated from one another; and
a control unit configured to drive the moving optical system based on the set cutting line and to cut a corneal tissue with laser light.

2. The ophthalmic laser surgery apparatus according to claim 1, wherein
the setting unit includes a storage unit configured to store a cutting pattern having a cutting line according to a predetermined rule, and
the setting unit sets, based on the cutting pattern, a cutting line such that a width of the cut tissue is equal to or less than a half of the diameter of the lenticular tissue.

3. The ophthalmic laser surgery apparatus according to claim 2, wherein the cutting pattern is a pattern having a cutting line configured such that the cut tissue is a spiral tissue.

4. The ophthalmic laser surgery apparatus according to claim 2, wherein the cutting pattern is a pattern having a cutting line configured such that both ends of the cut tissue are located on an outer periphery of the lenticular tissue and that the cut tissue has a zigzag-shape.

5. The ophthalmic laser surgery apparatus according to claim 2, wherein
the setting unit includes an input unit configured to input a width of an incisional wound formed on the cornea to take out the cut tissue to outside of the cornea, and a diameter of the lenticular tissue,
a width of the cut tissue which is cut based on the input width of the incisional wound is determined to have a value equal to or less than a half of the diameter of the lenticular tissue, and
the cutting line is set based on the determined width and the cutting pattern.
